(19) United States
(12) Patent Application Publication
(10) Pub. No.: US 2023/0104178 A1
(43) Pub. Date: Apr. 6, 2023

(54) SYSTEM AND METHOD FOR PERFORMING CONSUMER HEARING AID FITTINGS

(71) Applicant: Team IP Holdings, LLC, Fort Worth, TX (US)

(72) Inventors: Dan Mapes-Riordan, Wilmette, IL (US); Keith L. Davis, Salt Lake City, IL (US); Timothy D. Schnell, Del Mar, CA (US)

(21) Appl. No.: 17/959,452

(22) Filed: Oct. 4, 2022

Related U.S. Application Data
(60) Provisional application No. 63/252,819, filed on Oct. 6, 2021.

(51) Int. Cl. H04R 25/00; A61B 5/00; A61B 5/12

(52) U.S. Cl. CPC H04R 25/70; A61B 5/742; A61B 5/125; A61B 2560/0223

(57) ABSTRACT

Certain embodiments provide systems and methods for performing consumer hearing aid fittings. The system includes a computing device, an otoacoustic emissions (OAE) measurement device, a hearing aid, and a hearing database. The hearing database is configured to store customer hearing data. The OAE measurement device is configured to perform an OAE test to generate OAE test results. The computing device is communicatively coupled to the hearing database, the OAE measurement device, and the hearing aid. The computing device is configured to receive the OAE test results from the OAE measurement device, retrieve a subset of the hearing data from the hearing database, process the OAE test results and the subset of the hearing data to generate hearing aid fitting parameters, and upload the hearing aid fitting parameters to the hearing aid. The hearing aid is configured to apply the hearing aid fitting parameters to generate an acoustic output.

### CROSS-REFERENCE TO RELATED APPLICATIONS/INCORPORATION BY REFERENCE

The present application claims priority under 35 U.S.C. § 119(e) to provisional application Ser. No. 63/252,819 filed on Oct. 6, 2021, entitled “SYSTEM AND METHOD FOR PERFORMING CONSUMER HEARING AID FITTINGS.” The above referenced provisional application is hereby incorporated herein by reference in its entirety.

Gorga et al., “From laboratory to clinic: A large scale study of distortion product otoacoustic emissions in ears with normal hearing and ears with hearing loss,” Ear and Hearing, 18, 440-455, 1997, is incorporated by reference herein in its entirety.

Parker, “Identifying three otopathologies in humans,” Hearing Research, 398, 2020, is incorporated by reference herein in its entirety.

Belitz, et al., “A Machine Learning Based Clustering Protocol for Determining Hearing Aid Initial Configurations from Pure-Tone Audiograms,” INTERSPEECH 2019, 2325-2329, 2019, which is incorporated by reference herein in its entirety.

### FIELD

The present disclosure relates to hearing aids. More specifically, the present disclosure relates to a system and method that provides consumers with tools to perform hearing aid fittings.

### BACKGROUND

Hearing aids (HA) are typically customized for specific users by manufacturers and hearing care professionals (HCP). These customizations improve comfort and acoustic performance particular to a user's unique hearing impairment. The customizations include physical modifications to the device and configuration of electro-acoustic characteristics.

Personal sound amplification products (PSAP) are typically distributed directly to a consumer, without assistance of a hearing care professional. Customizations made available to the user are typically limited to basic adjustments, such as volume control, low resolution equalization, and program selection among pre-programmed generic fittings.

The distinction between hearing aids and personal sound amplification products is disappearing with new regulations, new modes of distribution, and new technological capabilities that bridge the gap between these former U.S. Food and Drug Administration (FDA) designations. For purposes of the present disclosure, personal sound amplification products are considered to be in the same class as hearing aids.

Remote control devices and smart-phone applications are currently available, which allow a user to make basic adjustments to the hearing aid device configuration, such as volume control, program selection, or basic equalization. Some applications also provide for remote communication between the user and a hearing care professional, where the hearing care professional can prepare and send a digital package of fitting information to the user's mobile device, which the user can then load into the hearing aid to change its electro-acoustic performance.

The traditional method of tuning hearing aid parameters to hearing loss of an individual uses a measurement of the individual's ability to detect tones at their hearing threshold (i.e., an audiogram). These measurements are traditionally made by an audiologist in a clinical setting. If a customer later finds they are not satisfied with their hearing aid parameters, their only recourse is to return to the audiologist's office for retuning.

Further limitations and disadvantages of conventional and traditional approaches will become apparent to one of skill in the art, through comparison of such systems with some aspects of the present disclosure as set forth in the remainder of the present application.

### SUMMARY

Certain embodiments of the present technology provide a system and method for providing consumers with tools to perform hearing aid fittings, substantially as shown in and/or described in connection with at least one of the figures.

These and other advantages, aspects and novel features of the present disclosure, as well as details of an illustrated embodiment thereof, will be more fully understood from the following description and drawings.

### BRIEF DESCRIPTION OF THE DRAWINGS

FIG. 1 illustrates a block diagram of an exemplary system configured to provide consumer tools for providing a hearing aid fitting, in accordance with embodiments of the present technology.

FIG. 2 illustrates an exemplary structure of a hearing database, in accordance with embodiments of the present technology.

FIG. 3A illustrates an exemplary flowchart of the operation of the otoacoustic emissions (OAE) user software, in accordance with embodiments of the present technology.

FIG. 3B is a continuation of the OAE user software flowchart of FIG. 3A, illustrating exemplary steps for entering user settings, in accordance with embodiments of the present technology.

FIG. 3C is a continuation of the OAE user software flowchart of FIG. 3A, illustrating an exemplary execution of a new OAE test, in accordance with embodiments of the present technology.

FIG. 3D is a continuation of the OAE user software flowchart of FIG. 3A, illustrating exemplary steps for reviewing OAE test results and the associated recommended hearing aid fitting, in accordance with embodiments of the present technology.

FIG. 3E is a continuation of the OAE user software flowchart of FIG. 3A, illustrating exemplary steps for selecting a hearing aid fitting based on the current OAE test and a database of previous tests, in accordance with embodiments of the present technology.

FIG. 4 illustrates an exemplary flowchart of a hearing aid fitting optimization procedure, in accordance with embodiments of the present technology.

FIG. 5 illustrates an exemplary graph showing a relationship between OAE signal-to-noise ratio (SNR) and audiogram hearing loss (HL), in accordance with embodiments of the present technology.

FIG. 6 illustrates an exemplary flowchart of the operation of the OAE measurement device firmware, in accordance with embodiments of the present technology.

### DETAILED DESCRIPTION

Embodiments of the present technology provide a system and method for providing consumers with tools to perform hearing aid fittings. Aspects of the present disclosure provide the technical effect of allowing a user to adjust hearing aid parameters in a guided fashion in their own environment. Various embodiments provide the technical effect of allowing users to measure their own otoacoustic emissions (OAEs). Certain embodiments provide the technical effect of combining OAE measurements with the knowledge of other users OAE measurements, audiograms, hearing speech in noise performance (e.g., QuickSIN), and hearing aid fitting parameters to suggest a preferred set of parameters for the current user. Aspects of the present disclosure provide the technical effect of predicting a hearing aid fitting of a user based solely on their OAE measurements using a database of measurements of settings of hearing aid users linked to OAE and audiogram measurements and hearing performance.

Research has shown there is good correlation between a person's audiogram and their OAE measurement. See e.g., Gorga et al., “From laboratory to clinic: A large scale study of distortion product otoacoustic emissions in ears with normal hearing and ears with hearing loss,” Ear and Hearing, 18, 440-455, 1997, which is incorporated herein by reference in its entirety. There is also accumulating evidence to show that OAEs are a better indicator of hearing speech in a noisy background (e.g. a noisy restaurant) than an audiogram. See e.g., Parker, “Identifying three otopathologies in humans,” Hearing Research, 398, 2020, which is incorporated by reference herein in its entirety. In addition, there exists a growing database of measurements of hearing aid users' settings linked to their OAE and audiogram measurements and their hearing performance.

Various embodiments provide an OAE measuring device used in conjunction with a computing device, such as a smart phone, PC, tablet, or the like, operable to receive the OAE measurement. The computing device is communicatively coupled to an extensive database of hearing measurements and hearing aid fittings, which is used to derive a useful hearing aid fitting based on the OAE measurement.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

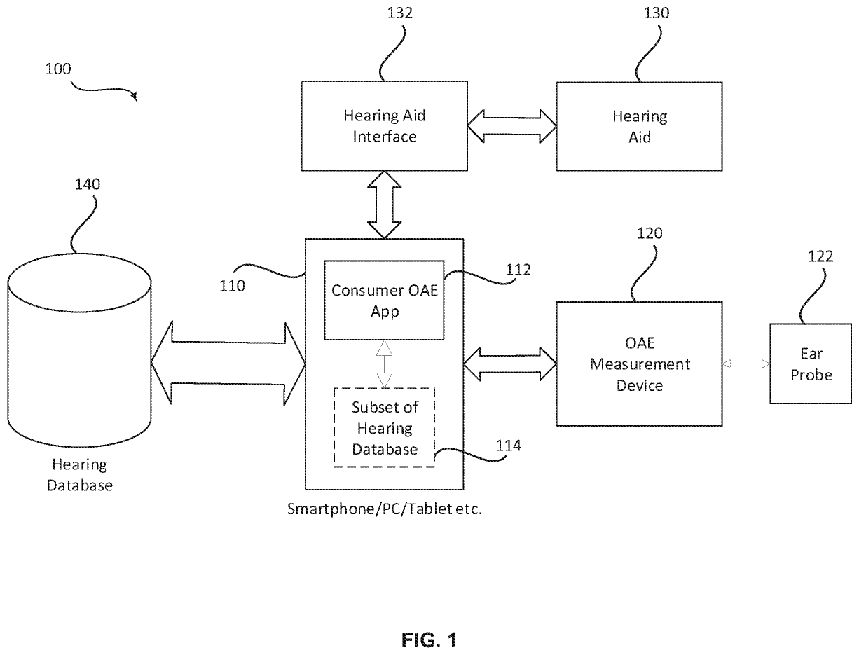

FIG. 1 illustrates a block diagram of an exemplary system 100 configured to provide consumer tools for providing a hearing aid fitting, in accordance with embodiments of the present technology. Referring to FIG. 1, the system 100 includes a computing device 110, a hearing aid 130, an OAE measurement device 120, and a hearing database 140. The hearing database 140 may exist centrally on another computer system or server, cloud-based, or the like. Additionally and/or alternatively, the hearing database 140 may exist locally (e.g., on the OAE measurement device 120 or the computing device 110). The hearing database 140 comprises hearing related parameters and is a common repository for user hearing related data. The hearing database 140 may be accessible to all of the hearing measurement and related systems.

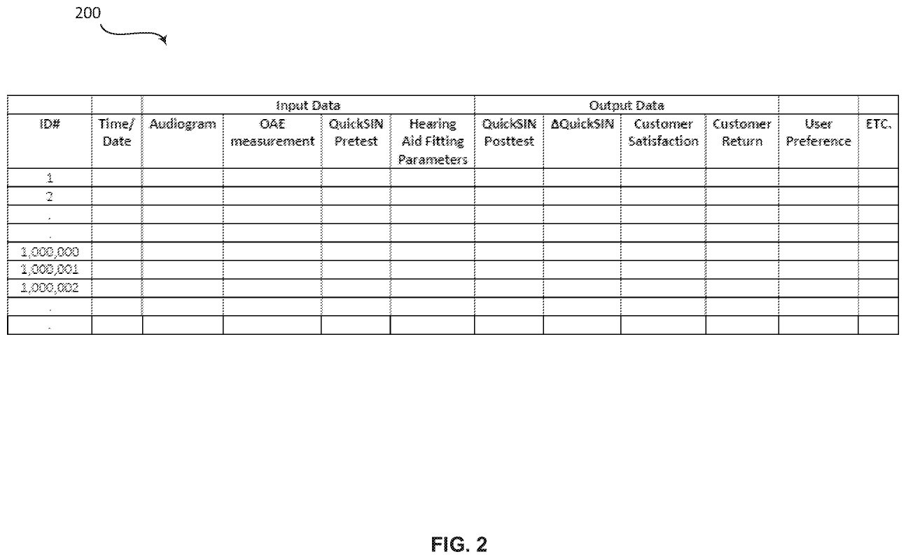

FIG. 2 illustrates an exemplary structure of a hearing database 200, in accordance with embodiments of the present technology. Referring to FIG. 2, each row represents an individual user with possible repeat tests. The columns represent the hearing test data for each user along with their hearing aid fitting, as well as output measures of hearing aid performance as quick speech-in-noise (QuickSIN) improvement, user satisfaction, customer returns, and the like. The characteristic of the database 200 may be of a common form (e.g., structured query language (SQL)) that allows many types of hearing systems to upload and query hearing related data. The first column (ID #) represents a unique user for each entry and each row is a hearing related data entry for a particular user. In various embodiments, the database 200 is anonymous to support privacy requirements. Specifically, no personal information is included in the database 120, and no real user can be identified by analysis of the data. Users can have multiple entries such as for different dates when measurement data was collected. Columns 2 to 5 are hearing measurements that have been made. The subsequent columns are outcome measures that relate to how well a set of hearing aid fitting parameters are valued by the user, such as satisfaction ratings, user preferences, customer return data, and the like.

Referring again to FIG. 1, the OAE measurement device 120 is a portable test system that includes an integrated or detachable ear probe 122 for making OAE measurements. The OAE measurement device 120 is communicatively coupled to the computing device 110 by a wireless (e.g., Bluetooth) or wired (e.g., USB) connection. The OAE measurement device 120 comprises suitable logic, circuitry, interfaces, and/or code configured to retrieve a test protocol, test for a seal of the ear probe 122, test for noise in the ear, calibrate output levels, output test tones, and measure received OAEs. The test protocol may define the frequencies being measured, the output level of the test tones, the duration of the test tones, and the like. The test protocol may be retrieved by OAE measurement device 120 and/or provided to the OAE measurement device 120 by the computing device 110 based on user settings (e.g., patient age), a user selection, or the like. The OAE measurement device 120 may be configured to detect noise in an ear, such as internal noise (e.g., patient chewing) or external noise (e.g., environmental noise). The OAE measurement device 120 may be configured to detect a drop in low frequency levels indicating a lack of probe seal. The OAE measurement device 120 may be configured to calibrate the test tones prior to conducting an OAE test. The OAE measurement device 120 may be configured to conduct an OAE test, store and/or transmit test results of a successful test, and store and/or transmit error flags of an unsuccessful test (e.g., due to lack of probe seal and/or detected noise in ear).

The hearing aid 130 comprises one or more microphones, one or more receivers, memory, one or more processors, and communication connections. The one or more microphones are configured to receive sound exterior to an ear canal. The microphones convert the sound to electrical signals and provide the electrical signals to the one or more processors. The one or more processors modify the sound level by applying hearing aid parameters retrieved from memory and/or received from the computing device 110. The one or more processors pass the electrical signals having the modified sound level to the receiver. The receiver converts the electrical signals to sound, which is communicated from the receiver to a user's ear canal. The memory, one or more processors, and communication connections of the hearing aid 130 may share various characteristics with the memory, one or more processors, and communication connections as described below with respect to the computing device 110. The hearing aid 130 comprises a hearing aid interface 132 that comprises suitable logic, circuitry, interfaces, and/or code that is operable to transmit and receive information with the computing device 110. The hearing aid interface 132 may comprise a hearing aid docking station, a wired interface, and/or a wireless interface (e.g., transceiver), for example.

The computing device 110 may comprise, for example, a smart phone, a tablet computer, a personal computer, or any suitable electronic device capable of communication with the hearing aid 130, OAE measurement device 120, and hearing database 140 via wired or wireless connections, such as Bluetooth, BLE, short-range, long range, Wi-Fi, cellular, personal communication system (PCS), USB, or any suitable wired or wireless connection.

The computing device 110 may include a display, user input devices, a memory, one or more processors, one or more communication connections, and the like. The display may be any device capable of communicating visual information to a user. For example, a display may include a liquid crystal display, a light emitting diode display, and/or any suitable display. The display can be operable to display information from a software application, such as a consumer OAE application 112, or any suitable information. In various embodiments, the display may display information provided by the one or more processors, for example.

The user input device(s) may include a touchscreen, button(s), motion tracking, orientation detection, voice recognition, a mousing device, keyboard, camera, and/or any other device capable of receiving a user directive. In certain embodiments, one or more of the user input devices may be integrated into other components, such as the display, for example. As an example, user input device may include a touchscreen display.

The memory may be one or more computer-readable memories, for example, such as compact storage, flash memory, random access memory, read-only memory, electrically erasable and programmable read-only memory and/or any suitable memory. The memory may include databases, libraries, sets of information, or other storage accessed by and/or incorporated with the one or more processors, for example. The memory may be able to store data temporarily or permanently, for example. The memory may be capable of storing data generated by the one or more processors and/or instructions readable by the one or more processor, among other things. In various embodiments, the memory stores information related to a consumer OAE application 112 and a subset of the hearing database 114, for example.

The communication connection(s) allow communication between the computing device and other external systems, such as the hearing aid 130, the hearing database 140, and the OAE measurement device 120, for example. The communication connection(s) may include wired and/or wireless interfaces. The wireless interfaces may include transceivers, such as Bluetooth, short-range, long range, Wi-Fi, cellular, personal communication system (PCS), or any suitable transceiver.

The one or more processors may be one or more central processing units, microprocessors, microcontrollers, and/or the like. The one or more processors may be an integrated component, or may be distributed across various locations, for example. The one or more processors may be capable of executing a software application, receiving input information from a user input device and/or communication connection(s), and generating an output displayable by a display, among other things. The one or more processors may comprise suitable logic, circuitry, interfaces, or code configured to control the OAE measurement device 120, query and update the hearing database 140, identify hearing aid fitting parameters based on the OAE measurement, and upload the identified hearing aid fitting parameters to the user's hearing aid 130. In certain embodiments, the one or more processors may communicate via communication connection(s) with the OAE measurement device 120 to perform measurement control and obtain OAE measurements. In various embodiments, the one or more processors may communicate via communication connection(s) with the hearing database 140 to perform measurement queries and store obtained measurements, for example. In an exemplary embodiment, the one or more processors may communicate via communication connection(s) with the hearing aid 130 to upload the hearing aid fitting parameters. For example, the one or more processor may send hearing aid fitting parameters selected based on the OAE measurement and the hearing database queries to the hearing aid devices 130.

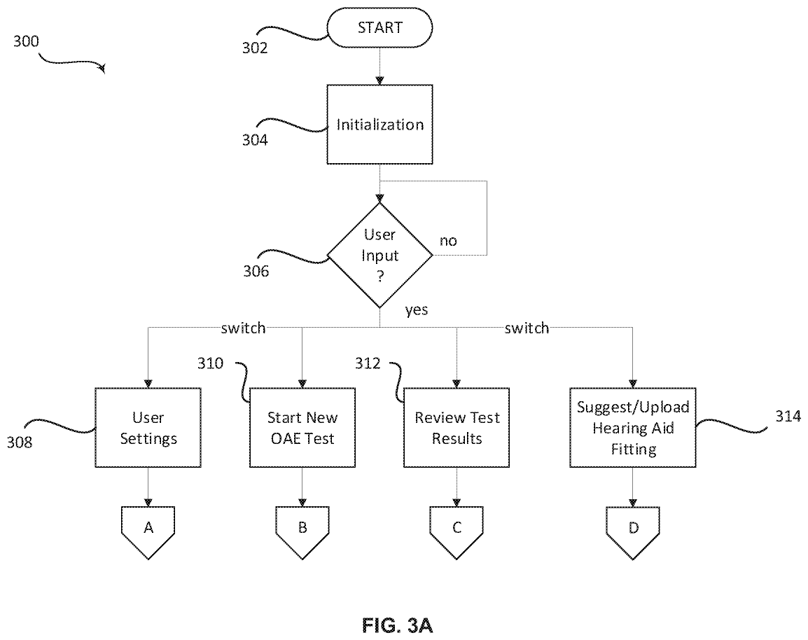

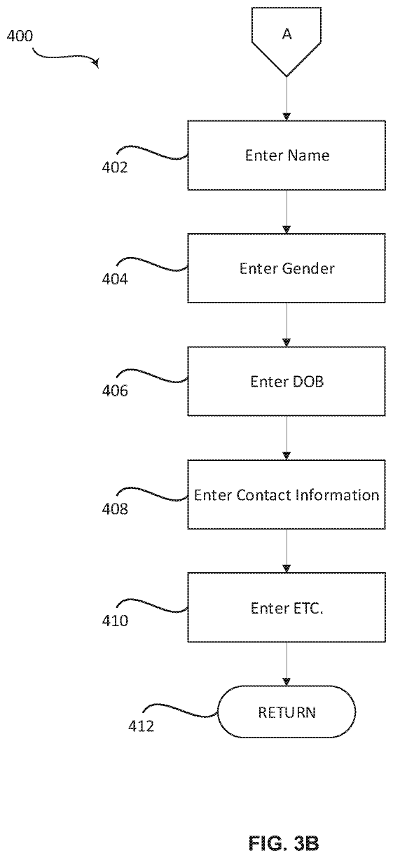

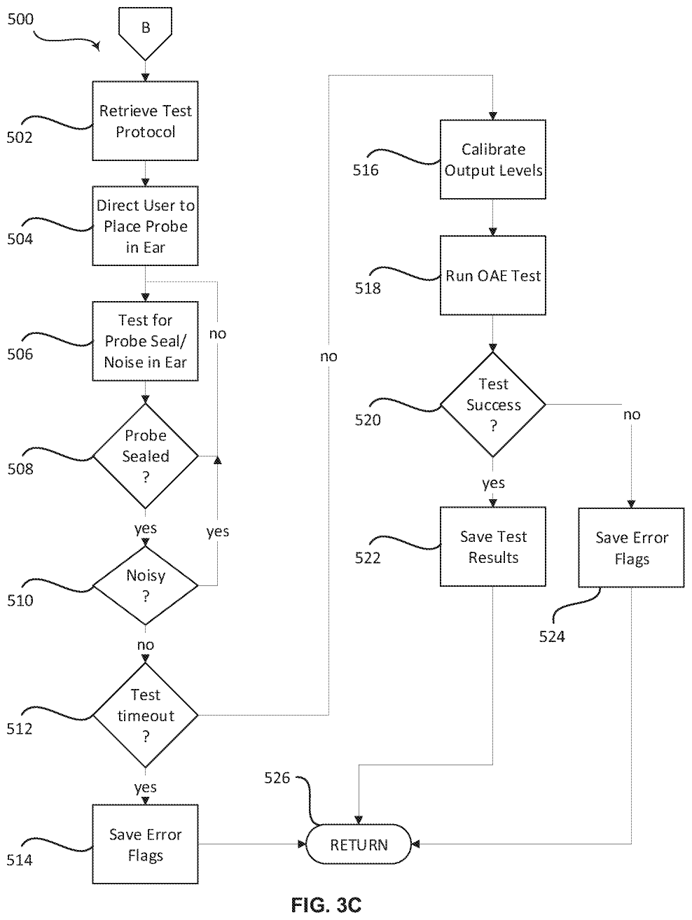

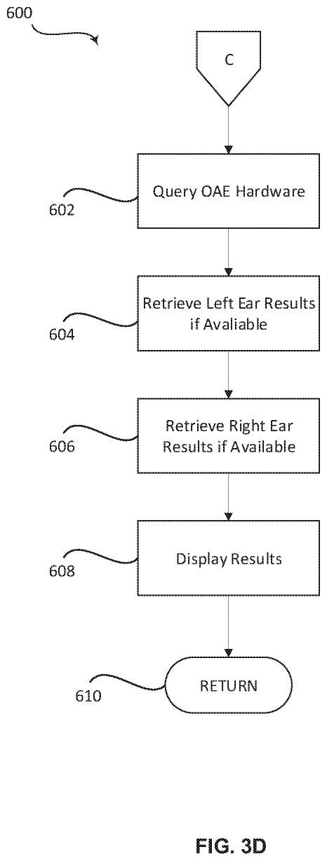

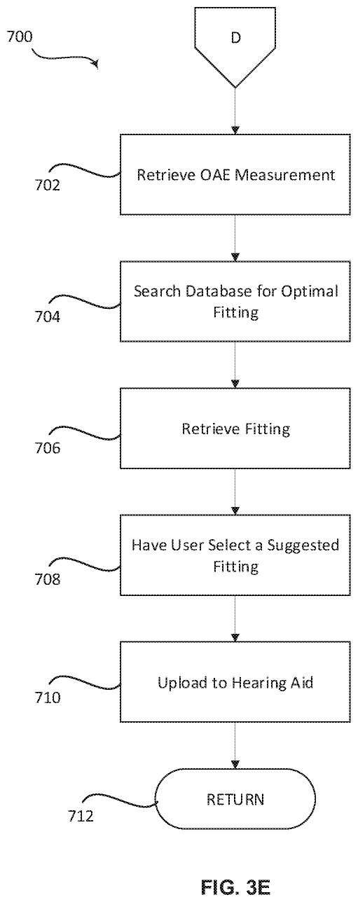

FIG. 3A illustrates an exemplary flowchart 300 of the operation of the otoacoustic emissions (OAE) user software 112, in accordance with embodiments of the present technology. Referring to FIG. 3A, the computing device 110 may be powered on and the OAE user software 112 selected at step 302. At step 304, the OAE user software 112 is initialized. After initialization, the computing device 110 awaits a user input at step 306. The user input may be an instruction to view/modify settings at step 308, start a new OAE test at step 310, review OAE test results at step 312, or suggest and upload a hearing aid fitting at step 314.

FIG. 3B is a continuation of the OAE user software flowchart 300 of FIG. 3A, illustrating exemplary steps 400 for entering user settings, in accordance with embodiments of the present technology. Referring to FIG. 3B, in response to a user input instructing the OAE user software 112 to view/modify settings at step 308 of FIG. 3A, the user may enter or review settings that identify the user. For example, at step 402, the user may enter a name. At step 404, the user may enter a gender. At step 406, the user may enter a date of birth (DOB). At step 408, the user may enter contact information. At step 410, the user may enter any additional suitable information to identify the user. The settings data may be tagged with each measurement and become part of the test record stored in the hearing database 140, 200 as illustrated in FIGS. 1 and 2. In various embodiments, the setting data may be used by the computing device 110 and/or OAE measurement device 120 to select a test protocol. For example, a test protocol may be selected based on an age of the user, among other things. At step 412, the process may return to step 306 of FIG. 3A to await a user input.

FIG. 3C is a continuation of the OAE user software flowchart 300 of FIG. 3A, illustrating an exemplary execution 500 of a new OAE test, in accordance with embodiments of the present technology. Referring to FIG. 3C, in response to receiving a user input instructing the OAE user software 112 to start a new OAE test at step 310 of FIG. 3A, the computing device 110 may retrieve a test protocol and upload the test protocol to the OAE measurement device 120 at step 502. Additionally and/or alternatively, the OAE measurement device 120 may retrieve a test protocol in response to instructions from the computing device 110 at step 502. The test protocol may be retrieved by OAE measurement device 120 and/or provided to the OAE measurement device 120 by the computing device 110 based on user settings (e.g., patient age), a user selection, or the like. At step 504, the user is directed to place the ear probe 122 of the OAE measurement device 120 into an ear of the user. At step 506, the OAE measurement device 120 tests for a seal. For example, a seal of the ear probe 122 may be detected by measuring a low frequency spectrum in the ear. The low frequency level is relatively high when there is a seal and drops to a lower level when there is a leak. At step 508, if a leak is detected, the process returns to step 506 to continue testing the probe seal. If a seal is detected, the process continues to step 510. At step 506, the OAE measurement device 120 also tests for noise in the ear. If noise is detected at step 510, the process returns to step 506 to continue testing for noise in the ear. If noise is not detected in the ear at step 510, the process proceeds to step 512. The OAE measurement device 120 will not start a test until a seal is detected and the noise is sufficiently low to measure OAEs. At step 512, the test aborts after a certain duration (e.g., 30 seconds) if the probe 122 cannot be sealed and the noise kept to a low level. If the test aborts at step 512, error flags indicating the reasons for the aborted test are saved and/or transmitted to the computing device 110 at step 514. The process 500 then proceeds to step 526, where the process 500 returns to step 306 of FIG. 3A to await a user input.

At step 512, if the probe 122 is sealed and the noise kept to a low level, the process proceeds to step 516 where the output levels of the test tones are calibrated by the OAE measurement device 120. At step 518, the OAE measurement device 120 performs the OAE test by emitting test tones via the ear probe 122 and measuring the OAE response. At step 520, the test is deemed successful if the background noise level is kept low and probe seal is maintained during the test. If the test is deemed unsuccessful at step 520, error flags indicating the reasons for the unsuccessful test are saved and/or transmitted to the computing device 110 at step 524. The process 500 than proceeds to step 526, where the process 500 returns to step 306 of FIG. 3A to await a user input. If the test is deemed successful at step 520, the results of the test are saved in the OAE measurement device 120, transmitted to the computing device 110, and/or uploaded to the hearing database 140. In various embodiments, the test result record may be saved locally if there is not an active connection to the hearing database 140, with the results later uploaded when an active connection is detected. The process 500 than proceeds to step 526, where the process 500 returns to step 306 of FIG. 3A to await a user input.

FIG. 3D is a continuation of the OAE user software flowchart 300 of FIG. 3A, illustrating exemplary steps 600 for reviewing OAE test results and the associated recommended hearing aid fitting, in accordance with embodiments of the present technology. Referring to FIG. 3D, in response to receiving a user input instructing the OAE user software 112 to review test results at step 312 of FIG. 3A, the computing device 110 may query the OAE measurement device 120, the hearing database 140, and/or the subset of hearing database 114 at step 602 and retrieve left ear results at step 604 and/or right ear results at step 606. The retrieved results may be presented at a display of the computing device 110 at step 608, giving the user the ability to review a completed OAE test stored in the OAE measurement device 120, the hearing database 140, and/or the computing device 110. At step 610, the process 600 returns to step 306 of FIG. 3A to await a user input.

FIG. 3E is a continuation of the OAE user software flowchart 300 of FIG. 3A, illustrating exemplary steps 700 for selecting a hearing aid fitting based on the current OAE test and a database 114, 140 of previous tests, in accordance with embodiments of the present technology. Referring to FIG. 3E, in response to receiving a user input instructing the OAE user software 112 to suggest and upload a hearing aid fitting at step 314 of FIG. 3A, the computing device 110 may retrieve a most recent OAE measurement at step 702. For example, the most recent OAE measurement may be stored at the computing device 110, the OAE measurement device 120, and/or the hearing database 140. At step 704, the hearing database 140 and/or the subset of the hearing database 114 is searched for an optimal fitting based on the OAE measurement. The OAE measurement forms the input into an algorithm configured to identify an optimal hearing aid fitting with a similar OAE input. The algorithm, executed by the computing device 110 or hearing database 140, queries the data in the hearing database 140 and/or the subset of the hearing database 114 using a statistical method to identify the optimal hearing aid fitting. For example, the algorithm may analyze the hearing data to identify a correlation or regression relationship between OAE data and hearing aid parameters for the subset of data from satisfied customers. As another example, the algorithm may analyze the hearing data to identify hearing aid fitting parameters that produced a largest improvement in QuickSIN scores for a given OAE data.

In various embodiments, machine-learning techniques may be employed to obtain the optimum hearing aid fitting from OAE data at step 704. For example, using supervised machine learning, the output measures of delta QuickSIN scores, customer satisfaction ratings, and/or user preferences could be used with the audiogram and OAE input data to train a neural network to predict the optimal hearing aid fitting. It has also been shown how to use unsupervised learning to find natural clusters of fittings from audiograms. See e.g., Belitz, et al., “A Machine Learning Based Clustering Protocol for Determining Hearing Aid Initial Configurations from Pure-Tone Audiograms. INTERSPEECH 2019, 2325-2329, 2019, which is incorporated by reference herein in its entirety. OAE data could be included in this clustering analysis to improve this technique. Note that in all of these techniques, the algorithm and predictions are dynamic as the data set increases with time, correspondingly increasing optimum hearing aid identification performance with time. Certain embodiments may be used in conjunction with interactive fine-tuning adjustment techniques, where an optimum prediction fitting, based on OAE and other objective measures, may be further personalized using an interactive fine-tuning adjustment system.

At step 706, the optimal fitting is retrieved by the computing device 110. The computing device 110 may present the retrieved fitting at a display system of the computing device 110 for user review. At step 708, the computing device 110 receives a user selection confirming application of the suggested fitting. At step 710, in response to the user selection, the fitting is uploaded by the computing device 110 to the hearing aid 130 via the hearing aid interface 132. At step 712, the process 700 returns to step 306 of FIG. 3A to await a user input.

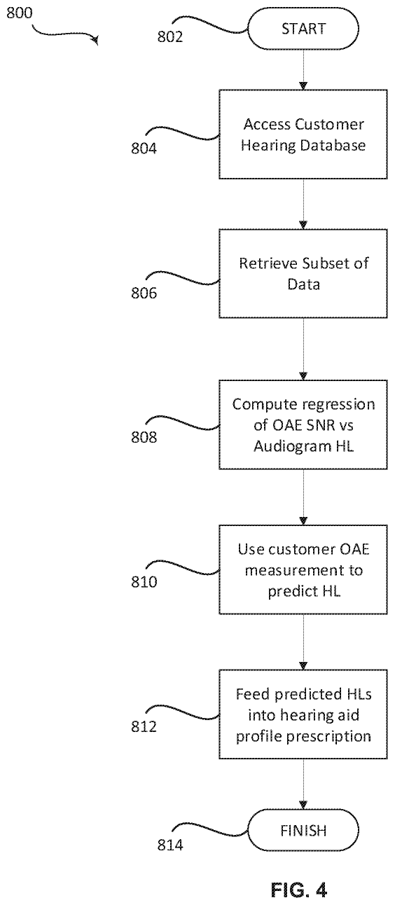

FIG. 4 illustrates an exemplary flowchart 800 of a hearing aid fitting optimization procedure, in accordance with embodiments of the present technology. The hearing aid optimization procedure of FIG. 4 is an exemplary embodiment of searching the hearing database 140 and/or subset of the hearing database 114 at step 704 of FIG. 3E as described above. The process 800 begins at step 802 and the hearing database 140 and/or the subset of the hearing database 114 is accessed at step 804. At step 806, a subset of the data to calculate the fitting is retrieved from the hearing database 140 and/or the subset of the hearing database 114. For example, only results with signal-to-noise ratios (SNRs) greater than zero could be used. As another example, the results may be filtered to include only data from previous customers whose product satisfaction scores are greater than or equal to satisfied. Additionally and/or alternatively, an age range could be used to filter the results. The resulting dataset comprises OAE level and noise floor values along with the corresponding audiogram hearing loss (HL) obtained from previous customers across the frequency range of interest.

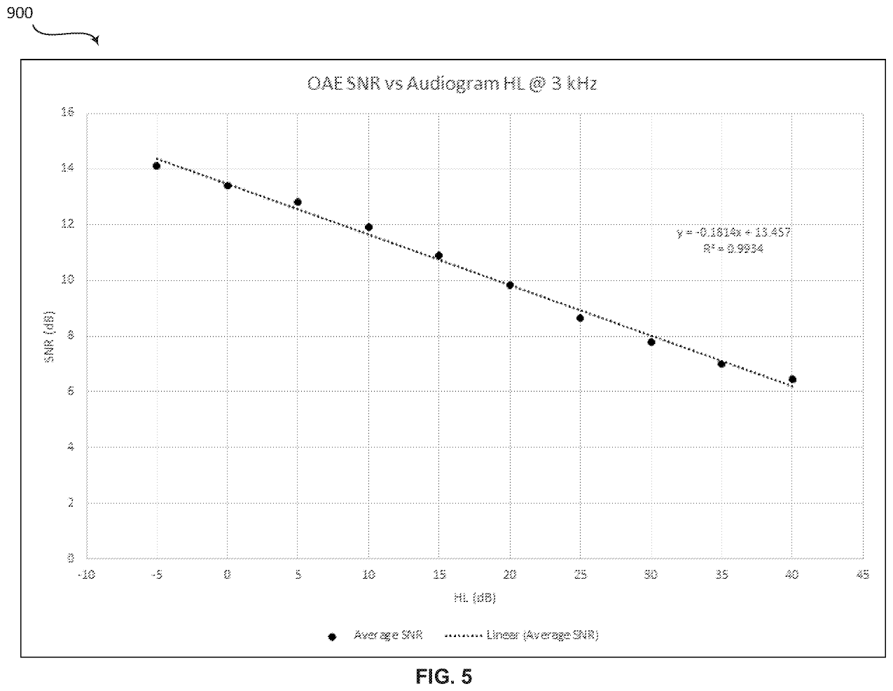

At step 808, a relationship between the OAE values and audiogram HLs is computed. For example, the relationship between the OAE values and audiogram HLs may be computed by applying a linear regression equation. In various embodiments, the computations may be provided at the hearing database 140 (i.e., a remote server) and/or on the user's computing device 110. FIG. 5 illustrates an exemplary graph 900 showing a relationship between OAE signal-to-noise ratio (SNR) and audiogram hearing loss (HL), in accordance with embodiments of the present technology. Referring to FIG. 5, the graph 900 and equation relating the average audiogram and OAE SNR values is illustrated at 3 kHz from a dataset of approximately 40,000 customer measurements. The graph 900 shows a strong linear relationship between OAE and HL values down to 40 dB HL. Similar equations would be derived for every frequency of interest. In an exemplary embodiment, the measured OAE levels may be applied rather than OAE SNR. In certain embodiments, multiple dimension correlations using QuickSIN and/or customer rating data may be applied. In a representative embodiment, correlations between spectral bands could also be included to improve the fitting relationship between OAEs and audiogram HLs. At step 810, the current user's OAE measurement data is applied to predict the audiogram HL values using the linear regression equations. At step 812, the predicted values are applied in the hearing aid profile prescription. At step 814, the process 800 is finished and may return to step 706 of FIG. 3E where the fitting is retrieved by the computing device 110.

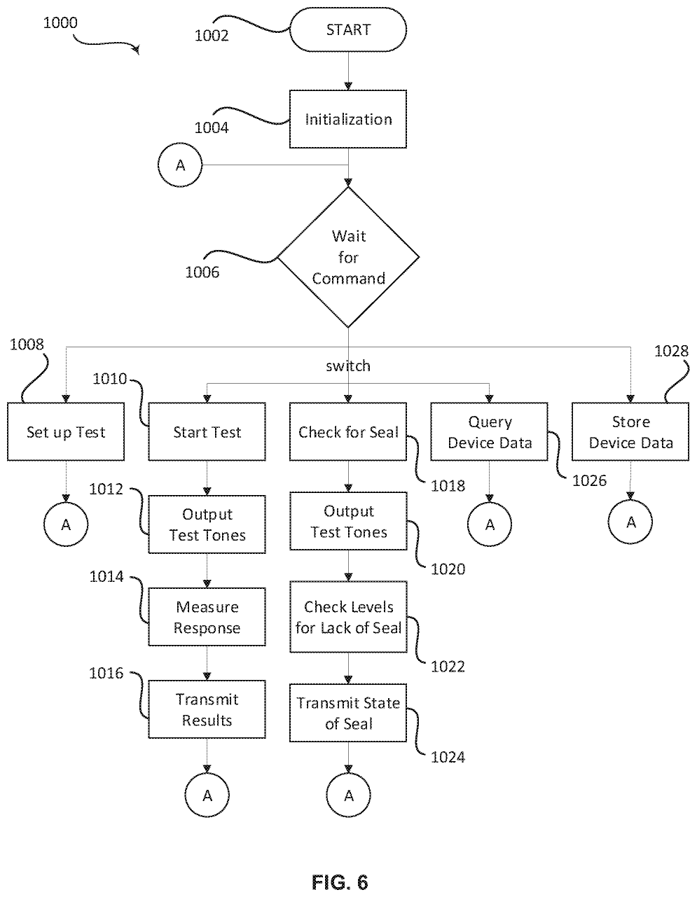

FIG. 6 illustrates an exemplary flowchart 1000 of the operation of the OAE measurement device 120 firmware, in accordance with embodiments of the present technology. Referring to FIG. 6, at steps 1002 and 2004 the OAE measurement device 120 is powered on and initialized. At step 1006, the OAE measurement device waits for commands from the OAE user software 112 of the computing device 110. The set of commands may include test parameter setup at step 1008, performing a seal check at steps 1018-1024, performing the OAE test at steps 1010-1016, receiving queries from the computing device 110 regarding the test results and/or protocol at step 1026, and storing device data at the OAE measurement device (e.g., test results and protocol information), among other things. Specifically, at step 1008, the OAE measurement device 120 may set up an OAE test in response to a command from the OAE user software 112 of the computing device 110 based on user settings and/or protocol information received from the computing device 110. For example, the OAE measurement device 120 may retrieve a protocol and calibrate the output levels for an OAE test. At step 1018, in response to a command from the OAE user software 112 of the computing device 110 to perform a seal check, the OAE measurement device 120 checks for a seal of the ear probe 122 by outputting test tones at step 1020, checking frequency levels at step 1022, and transmitting the state of seal to the computing device at step 1024. At step 1010, in response to a command from the OAE user software 112 to start an OAE test, the OAE measurement device 120 outputs test tones at step 1012, measures the OAE response at step 1014, and transmits and/or stores the results to the OAE measurement device 120, computing device 110, and/or the hearing database 140. At step 1026, the OAE measurement device 120 may receive a command from the OAE user software 112 querying the OAE measurement results stored at the OAE measurement device and/or other OAE measurement device data, such as protocol information and the like. At step 1028, the OAE measurement device 120 receives a command to store device data, such as OAE measurement results, protocol information, and the like.

Aspects of the present disclosure provide a system 100 and method 300, 400, 500, 600, 700, 800, 1000 for providing consumers with tools to perform hearing aid fittings. The system 100 comprises a computing device 110, an otoacoustic emissions (OAE) measurement device 120, a hearing aid 130, and a hearing database 140. The hearing database 140 is configured to store hearing data for a plurality of customers. The OAE measurement device 120 is configured to perform an OAE test to generate OAE test results. The computing device 110 is communicatively coupled to the hearing database 140, the OAE measurement device 120, and the hearing aid 130. The computing device 110 is configured to receive the OAE test results from the OAE measurement device 120. The computing device 110 is configured to retrieve at least a subset of the hearing data from the hearing database 140. The computing device 110 is configured to process the OAE test results and the at least the subset of the hearing data to generate hearing aid fitting parameters. The computing device 110 is configured to upload the hearing aid fitting parameters to the hearing aid 130. The hearing aid 130 configured to apply the hearing aid fitting parameters to generate an acoustic output.

In an exemplary embodiment, the hearing data comprises customer OAE measurements, customer audiogram hearing losses (HLs), and customer hearing aid fitting parameters for the plurality of customers. In a representative embodiment, the computing device 110 is configured to process the OAE test results and the at least the subset of the hearing data by computing a relationship between the OAE test results and the customer audiogram HLs. In various embodiments, the computing device 110 is configured to apply a linear regression equation to compute the relationship between the OAE test results and the customer audiogram HLs. In certain embodiments, the OAE test results comprises an OAE signal-to-noise ratio (SNR). The linear regression equation is applied to compute the relationship between the OAE SNR and the customer audiogram HLs. In an exemplary embodiment, the hearing data further comprises customer quick speech-in-noise (QuickSIN) improvement and customer rating data. In a representative embodiment, the computing device 110 is configured to generate the hearing aid fitting parameters based at least in part on one or both of the customer QuickSIN improvement and the customer rating data. In various embodiments, the computing device 110 is configured to display the OAE test results. In certain embodiments, the computing device 110 is configured to display the hearing aid fitting parameters. In an exemplary embodiment, the computing device 110 is configured to prompt a user selection of the hearing aid fitting parameters. The computing device 110 is configured to upload the hearing aid fitting parameters to the hearing aid 130 in response to the user selection of the hearing aid fitting parameters.

Various embodiments provide a system 100 and method 300, 400, 500, 600, 700, 800, 1000 for providing consumers with tools to perform hearing aid fittings. The method 300, 400, 500, 600, 700, 800, 1000 comprises performing 310, 500, 518, 1010-1014, by an otoacoustic emissions (OAE) measurement device 120, an OAE test to generate OAE test results. The method 300, 400, 500, 600, 700, 800, 1000 comprises receiving 602-606, 702 1016, 1026, by a computing device 110 communicatively coupled to the OAE measurement device 120, the OAE test results from the OAE measurement device 120. The method 300, 400, 500, 600, 700, 800, 1000 comprises retrieving 704, 806, by the computing device 120, at least a subset of hearing data from a hearing database 140 communicatively coupled to the computing device 110. The hearing database 140 comprises the hearing data 200 for a plurality of customers. The method 300, 400, 500, 600, 700, 800, 1000 comprises processing 704, 808, 810, by the computing device 110, the OAE test results and the at least the subset of the hearing data to generate hearing aid fitting parameters. The method 300, 400, 500, 600, 700, 800, 1000 comprises uploading 314, 710, by the computing device 110, the hearing aid fitting parameters to a hearing aid 130 communicatively coupled to the computing device 110. The method 300, 400, 500, 600, 700, 800, 1000 comprises applying, by the hearing aid 130, the hearing aid fitting parameters to generate an acoustic output.

In a representative embodiment, the method 300, 400, 500, 600, 700, 800, 1000 comprises dynamically updating 522, 1016 the hearing database 140 with the OAE test results. In various embodiments, the hearing data 200 comprises customer OAE measurements, customer audiogram hearing losses (HLs), and customer hearing aid fitting parameters for the plurality of customers. The processing 704, 808, 810 the OAE test results and the at least the subset of the hearing data comprises computing 808 a relationship 900 between the OAE test results and the customer audiogram HLs. In certain embodiments, the computing 808 the relationship 900 between the OAE test results and the customer audiogram HLs comprises applying a linear regression equation. In an exemplary embodiment, the OAE test results comprises an OAE signal-to-noise ratio (SNR). The applying the linear regression equation computes 808 the relationship 900 between the OAE SNR and the customer audiogram HLs. In a representative embodiment, the hearing data 200 comprises customer otoacoustic emissions (OAE) measurements, customer audiogram hearing losses (HLs), customer hearing aid fitting parameters, customer quick speech-in-noise (QuickSIN) improvement, and customer rating data. In various embodiments, the generating 704, 808, 810, by the computing device 110, the hearing aid fitting parameters is based at least in part on one or both of the customer QuickSIN improvement and the customer rating data. In certain embodiments, the method 300, 400, 500, 600, 700, 800, 1000 comprises displaying 608, by the computing device 110, the OAE test results. In an exemplary embodiment, the method 300, 400, 500, 600, 700, 800, 1000 comprises displaying 708, by the computing device 110, the hearing aid fitting parameters. In a representative embodiment, the method 300, 400, 500, 600, 700, 800, 1000 comprises prompting 708, by the computing device 110, a user selection of the hearing aid fitting parameters. The uploading 314, 710 the hearing aid fitting parameters to the hearing aid 130 is performed in response to the user selection of the hearing aid fitting parameters.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for providing consumers with tools to perform hearing aid fittings.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A system comprising:
a hearing database configured to store hearing data for a plurality of customers;
an otoacoustic emissions (OAE) measurement device configured to perform an OAE test to generate OAE test results;
a computing device communicatively coupled to the hearing database, the OAE measurement device, and a hearing aid, the computing device configured to:
receive the OAE test results from the OAE measurement device;
retrieve at least a subset of the hearing data from the hearing database;
process the OAE test results and the at least the subset of the hearing data to generate hearing aid fitting parameters; and
upload the hearing aid fitting parameters to the hearing aid; and
the hearing aid configured to apply the hearing aid fitting parameters to generate an acoustic output.

2. The system of claim 1, wherein the hearing data comprises customer OAE measurements, customer audiogram hearing losses (HLs), and customer hearing aid fitting parameters for the plurality of customers.

3. The system of claim 2, wherein the computing device is configured to process the OAE test results and the at least the subset of the hearing data by computing a relationship between the OAE test results and the customer audiogram HLs.

4. The system of claim 3, wherein the computing device is configured to apply a linear regression equation to compute the relationship between the OAE test results and the customer audiogram HLs.

5. The system of claim 4, wherein:
the OAE test results comprises an OAE signal-to-noise ratio (SNR), and
the linear regression equation is applied to compute the relationship between the OAE SNR and the customer audiogram HLs.

6. The system of claim 2, wherein the hearing data further comprises customer quick speech-in-noise (QuickSIN) improvement and customer rating data.

7. The system of claim 6, wherein the computing device is configured to generate the hearing aid fitting parameters based at least in part on one or both of the customer QuickSIN improvement and the customer rating data.

8. The system of claim 1, wherein the computing device is configured to display the OAE test results.

9. The system of claim 1, wherein the computing device is configured to display the hearing aid fitting parameters.

10. The system of claim 9, wherein:
the computing device is configured to prompt a user selection of the hearing aid fitting parameters, and
the computing device is configured to upload the hearing aid fitting parameters to the hearing aid in response to the user selection of the hearing aid fitting parameters.

11. A method comprising:
performing, by an otoacoustic emissions (OAE) measurement device, an OAE test to generate OAE test results;
receiving, by a computing device communicatively coupled to the OAE measurement device, the OAE test results from the OAE measurement device;
retrieving, by the computing device, at least a subset of hearing data from a hearing database communicatively coupled to the computing device, wherein the hearing database comprises the hearing data for a plurality of customers;
processing, by the computing device, the OAE test results and the at least the subset of the hearing data to generate hearing aid fitting parameters;
uploading, by the computing device, the hearing aid fitting parameters to a hearing aid communicatively coupled to the computing device; and
applying, by the hearing aid, the hearing aid fitting parameters to generate an acoustic output.

12. The method of claim 11, comprising dynamically updating the hearing database with the OAE test results.

13. The method of claim 11, wherein:
the hearing data comprises customer OAE measurements, customer audiogram hearing losses (HLs), and customer hearing aid fitting parameters for the plurality of customers, and
the processing the OAE test results and the at least the subset of the hearing data comprises computing a relationship between the OAE test results and the customer audiogram HLs.

14. The method of claim 13, wherein the computing the relationship between the OAE test results and the customer audiogram HLs comprises applying a linear regression equation.

15. The method of claim 14, wherein:
the OAE test results comprises an OAE signal-to-noise ratio (SNR), and
the applying the linear regression equation computes the relationship between the OAE SNR and the customer audiogram HLs.

16. The method of claim 11, wherein the hearing data comprises customer otoacoustic emissions (OAE) measurements, customer audiogram hearing losses (HLs), customer hearing aid fitting parameters, customer quick speech-in-noise (QuickSIN) improvement, and customer rating data.

17. The method of claim 16, wherein the generating, by the computing device, the hearing aid fitting parameters is based at least in part on one or both of the customer QuickSIN improvement and the customer rating data.

18. The method of claim 11, comprising displaying, by the computing device, the OAE test results.

19. The method of claim 11, comprising displaying, by the computing device, the hearing aid fitting parameters.

20. The method of claim 19, comprising prompting, by the computing device, a user selection of the hearing aid fitting parameters, wherein the uploading the hearing aid fitting parameters to the hearing aid is performed in response to the user selection of the hearing aid fitting parameters.
